(19) United States
(12) Patent Application Publication
(10) Pub. No.: US 2009/0010998 A1
(43) Pub. Date: Jan. 8, 2009

(54) Drug-delivery patch comprising a dissolvable layer and uses thereof

(76) Inventors: Kevin S. Marchitto, Place Golden, CO (US); Stephen T. Flock, Place Arvada, CO (US)

Correspondence Address: Benjamin Aaron Adler;ADLER & ASSOCIATES, 8011 Candle Lane, Houston, TX, 77071, US

(21) Appl. No.: 12/217,393

(22) Filed: Jul. 3, 2008

Related U.S. Application Data
(60) Provisional application No. 60/947,724, filed on Jul. 3, 2007.

(51) Int. Cl. A61K 9/70

(52) U.S. Cl. 424/449

(57) ABSTRACT

The present invention provides a drug-delivery patch having at least one dissolvable layer comprising an active material and an adhesive backing or cover. The present invention also provides a method of transdermally vaccinating an animal by ablating an area of the stratum corneum of the animal and applying the patch described herein to the area.

### CROSS-REFERENCE TO RELATED APPLICATION

This nonprovisional application claims benefit of provisional U.S. Ser. No. 60/947,724, filed Jul. 3, 2007, now abandoned.

### BACKGROUND OF THE INVENTION

1. Field of the Invention

The present invention relates generally to the fields of biomedical engineering, biochemistry and surgical procedures. More specifically, the present invention provides a device and methods using fast-dissolving films in laminates used to cover exposed dermis following stratum corneum ablation, or to cover wounds, for the purpose of delivering a pharmaceutical to the dermis or wounded tissue.

2. Description of the Related Art

Transdermal drug delivery systems suffer from several disadvantages. The skin is relatively impermeable to most drugs and medicaments as they cannot penetrate the relatively dry, keratinized outer layer, the stratum corneum (SC) [1]. As a result, there are currently available only a few drugs delivered transcutaneously. Successful examples include nicotine and fentanyl skin patches. Limitations of transdermal patch systems include manufacturability of multi-layer laminates, premature bursting of reservoir components, allergic responses to the adhesive and ability to manage release and dosaging of the drug into the tissues.

All of the skin patches approved by the U.S. Food and Drug Administration (FDA) that exist today are directed to be applied to intact dry skin. The release of drug from the patch is reliant on properties of the patch rather than skin. All of these patches are designed to deliver an active ingredient through intact stratum corneum, which is dry and somewhat lipophilic with very different chemical properties than the underlying, more hydrophilic tissue. Due to the impermeable nature of the SC, controlling the rate of permeation for transdermal patches is often a complicated matter. Most topically applied drugs permeate so slowly that their permeation properties often have to be enhanced in some way, usually with the use of penetration enhancers, such as alcohols.

Like its pharmaceutical counterparts, Transcutaneous Immunization (TCI) is also limited due to the inability of vaccines to penetrate the SC. TCI is a strategy for administering antigen to the skin that induces strong systemic and mucosal responses, does not use needles, may not require altering current vaccines or developing new vaccines, and is predicted to improve patient compliance and speed in administration [2]. Transcutaneous immunization can result in a 20-fold improved efficacy of a given vaccine [3,4]. Glenn et al. have shown that mucosal and systemic responses can be elicited to co-administered antigens with the use of enterotoxin or cholera toxin as adjuvants [5,6,7,8]. Humoral and cell-mediated responses have also been elicited through DNA immunization, in combination with chitosan and liposomes [9,10].

Needle-free vaccine delivery has many potential benefits over intramuscular (IM) and subcutaneous (SQ) delivery. Both IM and SQ delivery must be done by skilled clinicians and have deleterious side effects such as local pain, erythema and edema. In addition, many individuals fear injections (an estimated 7-22% of the general population have needle phobia) [11] which further limits compliance. A needle-free vaccination system would offer protection to more of the population.

When properly stimulated with adjuvant and antigen, transcutaneous immunization results in a robust humoral, mucosal and cellular response. Mucosal immune responses may complement systemic responses by protecting against pathogens at their point-of-entry. Studies suggest that skin may behave immunologically like a mucosal surface [12,13]. However, delivery of consistent and efficacious amounts of vaccine through the dry, keratinized stratum corneum layer of the skin to the subsurface dendritic cells is a challenge [14,15].

Perhaps the most significant difficulty associated with all transdermal drug delivery, including transcutaneous immunization is that the stratum corneum is largely impermeable to most topically applied pharmaceuticals [1]. Thus, significant research has been undergone in an effort to reduce or eliminate the barrier function of the stratum corneum thus allowing topically applied pharmaceuticals to permeate to the viable tissue and blood stream. The rate of permeation of pharmaceuticals and other medicaments through the dermis is greatly accelerated when the stratum corneum has been removed [1,16,17].

In the case of topical application of materials for the purpose of generating an immune response, dramatic enhancement of transcutaneous immunization is evident when the antigens are applied post-reduction of the stratum corneum [18]. One way to reduce or eliminate the barrier function of the stratum corneum is to reduce the stratum corneum itself. However, safe and efficacious removal of stratum corneum is difficult at best. Traditional methods of enhancing topical pharmaceutical uptake usually don't involve removal of the stratum corneum, but include iontophoresis, and most often are based on the optmization of drug and vehicle properties to enhance permeation through intact stratum corneum. Less common methods employ ultrasound or microporation. Other, more efficient methods include alteration or ablation of the stratum corneum using mechanical, optical, or thermal means [12].

Studies show that even a modest physical disruption of the stratum corneum can result in a dramatic improvement in the efficiency of TCI for a given dose [13,15,19-21]. These studies employed classic methods for stratum corneum disruption, including tape stripping and EKG prep pad abrasives. Using an EKG prep pad (emery paper), a 50-fold increase in the IgG titer was achieved. When hydration was used as a pretreatment, a 29-fold increase in the LT IgG response was achieved, indicating that physical disruption of the stratum corneum resulted in almost doubling of the increase in the LT IgG response using 87.5% less LT, an approximately 10-fold improvement in efficiency.

Normally, the skin acts as a barrier to environmental insults and maintains the subcellular layer in a state of homeostasis. When the SC is altered or removed, interstitial fluid may leak from the wound. In addition, when the skin becomes damaged, keratinocytes and Langerhans cells become activated [22]. These dendritic cells are believed to be critical in the induction of immunity to foreign antigens in the skin.

It is hypothesized that TCI applied antigen is processed by Langerhans cells in the skin, leading to the production of specific T-cell responses and systemic antibodies. It has been established that Langerhans cells are antigen presenting cells. Activated Langerhans cells increase their phagocytic activity and move from the skin into draining lymph nodes where they encounter foreign antigens and initiate immune responses [19]. They possess a constant level of transit from the skin to the draining lymph node, which is greatly amplified by contact sensitizers, lipopolysaccharides or cytokines such as TNF-α and IL-β with these cytokines in particular promoting migration of Langerhans cells [21]. They transport antigenic proteins [23], process them into immunogenic MHC-peptide complexes, present them to Ag-specific T cells in the T areas and, thus, efficiently elicit immune responses [18].

Activated keratinocytes also participate in a dermal response. They can synthesize a large number of cytokines involved in modulating the immune response [24]. In addition, keratinocytes can express intracellular adhesion molecules (ICAMs) and other adhesion molecules for various immune cells [25].

Stratum corneum disruption provides a route for antigens as large as 1 million Da to be delivered to the epidermis and elicit strong systemic immune responses [26] by reaching the dendritic cells (Langerhans and keratinocytes) which lie beneath the surface [27-29]. Although TCI research holds promise, widespread adoption of TCI will be limited until an effective, reproducible method of stratum corneum reduction is developed.

A recent study by Glenn and co-workers [30] demonstrated that physical disruption of the stratum corneum in humans can improve the efficiency of vaccine delivery and that the magnitude of stratum corneum disruption correlates with the immune response. In this study, the stratum corneum was disrupted using an electrocardiogram prep pad and hydration prior to patch application. In the Glenn et al. study, approximately 50% of the stratum corneum was removed after 15 passes of the abrasive pads. The approach and results underscore some of the problems associated with stratum corneum removal, and its difficulty. Classical methods of removing stratum corneum are variable with the results dependent largely on chance and the user's skill. Reproducibility and consistency are simply not possible, implying a significant variability in the tissues exposure to antigen is likely. This factor alone makes it unlikely that these methods would pass FDA scrutiny as an approved drug delivery method through the skin. Secondly, the methods, such as tape-stripping and abrasion using EKG pads, requiring 15 or more tape strips or passes of the abrasive pad, are irritating and painful to the patient.

Research demonstrates that anthrax vaccine could be delivered transdermally using abrasion and hydration. Glenn and co-workers [19] immunized mice by tape-stripping, or abrasion with pumice or emery, followed by skin hydration, and identified significant differences in the IgG response following treatment. Peachman and co-workers [15] showed that TCI with a purified AVA (called recombinant protective antigen, or rPA) induced a long-lasting neutralizing antibody titer in mice that was superior to IM injected controls. They further demonstrated a strong correlation between protection and the level of toxin-neutralizing antibodies in mice immunized with rPA and heat labile enterotoxin (HLT). The vaccine rPA, with Alhydrogel (aluminum hydroxide) as the adjuvant, recently underwent a Phase I trial (rPA102) which was designed to examine the safety and immune response of a range of doses of rPA102, and to compare them to AVA. The study showed an equivalent immune response to AVA, but with one tenth of the required adjuvant.

A recent study [18,31] demonstrated that, following stratum corneum removal, TCI using a skin patch impregnated with powderized rPA vaccine produced neutralizing antibody titers that were equivalent to those provided by IM administration. Moreover, this robust response was achieved without the use of adjuvants, which has the potential to reduce side effects. Although aluminum adjuvants are usually viewed as relatively safe, large-scale vaccination might gain better acceptance if no adjuvant, or a less reactogenic potent adjuvant, were used along with an improved immunization strategy [21]. TCI may result in the production of mucosal antibodies, which would be more desirable for conferring immunity to inhaled anthrax. Berry et al. [22] showed that TCI (achieved by razor shaving and acetone cleansing the skin) with CT and a Chlamydia antigen results in specific IgG and IgA.

The prior art is deficient in patch formulations designed for release of pharmaceuticals to skin that has been compromised by SC ablation or alteration. The prior art is also deficient in transdermal delivery patches that can deliver a metered dose. Specifically, the prior art is deficient in patch formulations that are designed to release pharmaceuticals after coming in contact with fluids expressed from skin that has become compromised in this regard. The present invention fulfills this longstanding need and desire in the art.

### SUMMARY OF THE INVENTION

The object of the invention it to deliver therapeutic or diagnostic material into tissue, most notably, the skin. The invention incorporates an excipient-pharmaceutical formulation (film) that is applied to skin following stratum corneum (SC) reduction, or another means of compromising the skin, such that interstitial fluid released from the now exposed underlying moist epidermis interacts with, and dissolves, the excipient, thereby releasing the active ingredient from the film. Optionally, the patch has additional non-degradable layers.

Another object of the invention is the delivery of a controlled dosage of a pharmaceutical substance or medicament through the dermis where the skin has been compromised such that the stratum corneum has been ablated. Still another object of the invention is pharmaceutical patch that enhances stability of the active component.

Another object of the invention is a drug-delivery patch incorporating at least one dissolving component for use in treating compromised skin, including skin wounds, whereby a layer which is proximal to the wound, or in contact with the wound, dissolves upon contact with fluids expressed from the wound, thereby releasing an active ingredient into the wound.

Another object of the invention is the collection of biomolecules from treatment site whereby fluids released from the site are absorbed into a film placed in contact with the site.

Thus, the present invention provide a drug-delivery patch, comprising at least one dissolvable layer, each layer comprising an active material and an adhesive backing or cover. Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows a drug-delivery patch with dissolvable layer.

FIGS. 2A-2D show different versions of a drug-delivery patch.

FIG. 3 shows a drug-delivery patch in contact with skin and structural details of the skin.

FIGS. 4A-4C show several different drug-delivery pathes with different components.

FIG. 5 shows a drug-delivery patch with a rupturable solvent reservoir.

FIG. 6 shows a drug-delivery patch with an iontophoretic delivery system incorporated within.

FIG. 7 shows a drug-delivery patch with a skin abrasion device incorporated within.

FIG. 8 shows dissolution of a dissolvable layer as a function of HPMC concentration.

FIG. 9 shows dissolution of a dissolvable layer with HPMC as a function of layer thickness.

FIG. 10 shows dissolution of a dissolvable layer as a function of HPC concentration.

FIG. 11 shows dissolution of a dissolvable layer with HPC as a function of layer thickness.

FIG. 12 shows immunization (TCI and IM) of mice, with a cellulose-type patch, and hemagluttinin (HA) administered with two different adjuvants.

FIG. 13 shows the neutralization assay results of immunization (TCI and IM) of mice, with a cellulose-type patch, and recombinent protective antigen (rPA) administered without adjuvant, or with gamma-interferon (γINF) or lipopolysaccharide (LPS).

FIG. 14 shows immunization (TCI and IM) of mice, with a dissolving-layer type patch, and recombinent protective antigen (rPA) administered without any adjuvant.

FIG. 15 shows immunization (TCI and IM) of mice, with a dissolving-layer type patch, and Norwalk virus-like-particles (nVLPs) administered without any adjuvant.

### DETAILED DESCRIPTION OF THE INVENTION

As used herein, the term “a” or “an”, when used in conjunction with the term “comprising” in the claims and/or the specification, may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

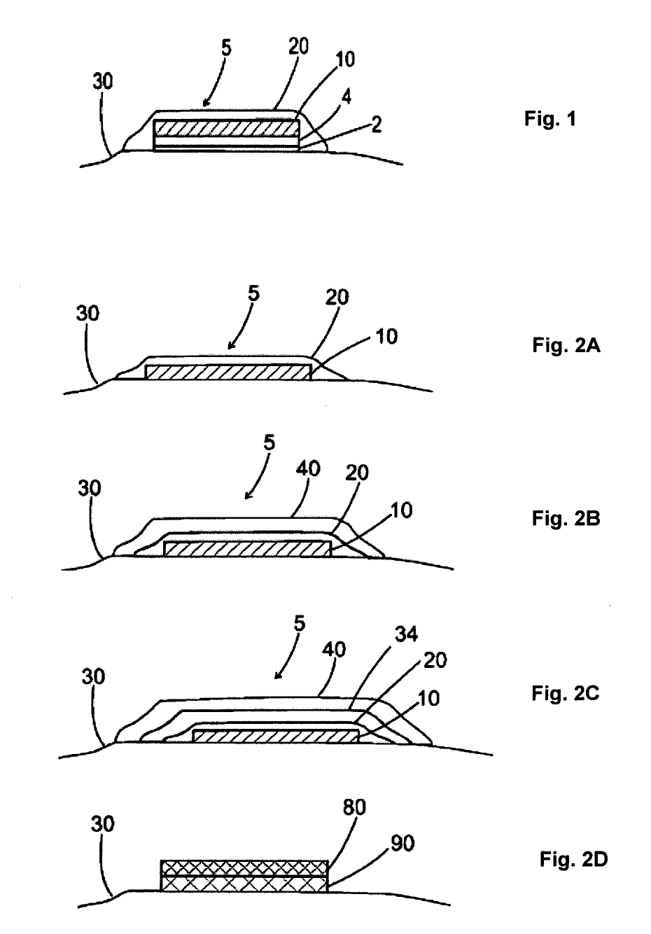

A basic design of the dissolving layer drug-delivery patch of the present invention is shown in FIG. 1. Here, the active material is incorporated into an excipient that forms a dissolvable layer 10; this layer 10 may be solid or semi-solid. The layer 10 is either held in intimate contact with the skin or other tissue 30 by an adhesive backing or cover 20. Optionally, the active material layer 10 is held against a membrane 4 which serves to control the rate at which the active material partitions from layer 10 into the skin or tissue 30. A protective liner 2 is shown in this figure, however, in practice this liner is removed from the patch before application to the tissue 30. When the patch is applied directly to wounded tissue, it may optionally consist only of a single layer 10, which dissolves upon contact with the moist wounded tissue. This layer 10 may have self-adhesive properties, depending on the nature of the excipient.

Other embodiments of the dissolving layer drug-delivery patch of the present invention are shown in FIGS. 2A-2D. In FIG. 2A, the active material is incorporated into an excipient, and forms a layer 10; this layer 10 may be solid or semi-solid. The layer 10 is held in intimate contact with the skin 30 by an adhesive backing 20. In FIG. 2B, an additional adhesive backing 40 holds the patch in contact with the skin 30 and serves to protect the patch from external mechanical insult or environmental elements. In FIG. 2C, another patch that additionally minimizes moisture loss to the ambient environment works with the addition of another layer 30 which has a very low mean water transmission rate (MTWR). Representative examples of such a material are aluminized polyester film or plastic food wrap. Optionally, in FIG. 2D, the layer 10 may be made up of two layers. One layer 80 contains the active material and a second dissolvable layer 90 is interposed between the active material 80 and skin 30. Such an arrangement may be necessary when the active material must be contained within a formulation that assures stability, but itself may not be dissolvable; an example of such a material may be a hydrogel. The layer 80 optionally may be a reservoir where the active material is held in a liquid form.

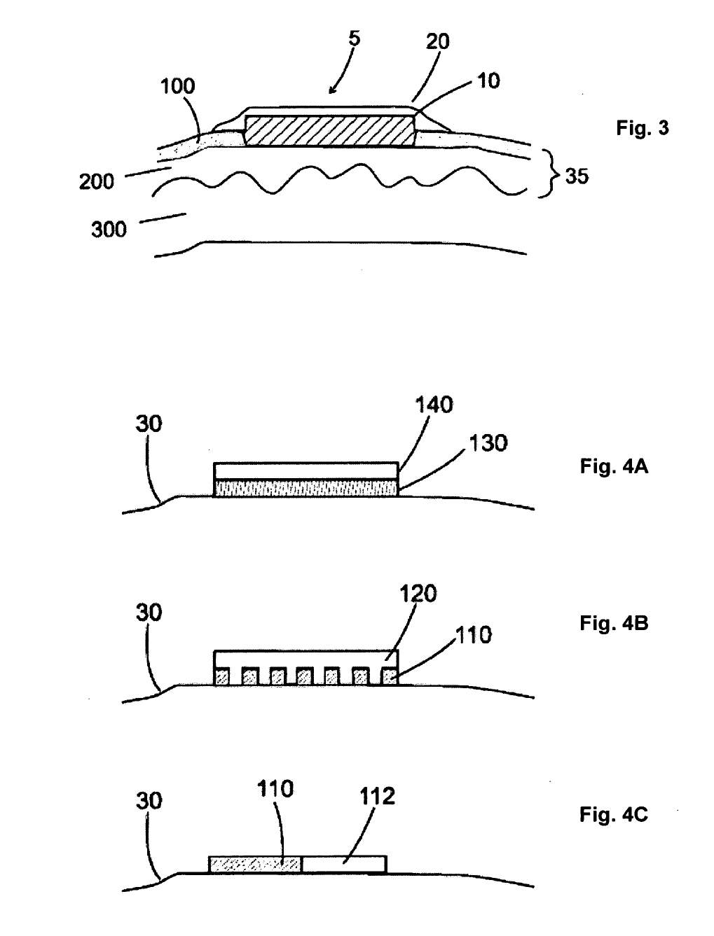

FIG. 3 illustrates the mechanism whereby the dissolution of the layer 10 is enhanced by the application of the patch 5 on stratum-corneum ablated skin 35. Here, the stratum corneum 100 is ablated, or altered so that it's permeable to the active material in layer 10, and the patch 5 is applied to the ablated skin 35. The moisture released from the epidermis 200 and sub-epidermal dermis 300 travels upwards through the ablated stratum corneum whereby it dissolves the layer 10 thus releasing the active material. Once released, the active material is free to diffuse downwards into the skin 35 whereby it performs it's therapeutic or diagnostic purpose.

FIGS. 4A-4C illustrate several different geometric arrangements of the dissolving layer(s). FIG. 4A, shows the dissolvable portion of a patch consisting of two layers, 130 and 140. Each layer may have a different active material. When applied to tissue, the first layer 130 dissolves first, thus releasing its active material, followed by dissolution of the second layer 140, which administers an active material that is beneficially administered after the active material from the first layer 130. An example of where this might be a beneficial arrangement is an adjuvant that could be in layer 130 and which immunogenically primes the skin prior to administration of the antigen, which is incorporated in layer 140. FIG. 4B shows another arrangement of two dissolving layers with active material that would allow for simultanous delivery of two different active materials, and then delivery of one active material (in layer 120) after some dissolution takes place. FIG. 4C, shows another arrangement of dissolving layers, 110 and 112, whereupon two different active materials could be delivered to the tissue at the same time.

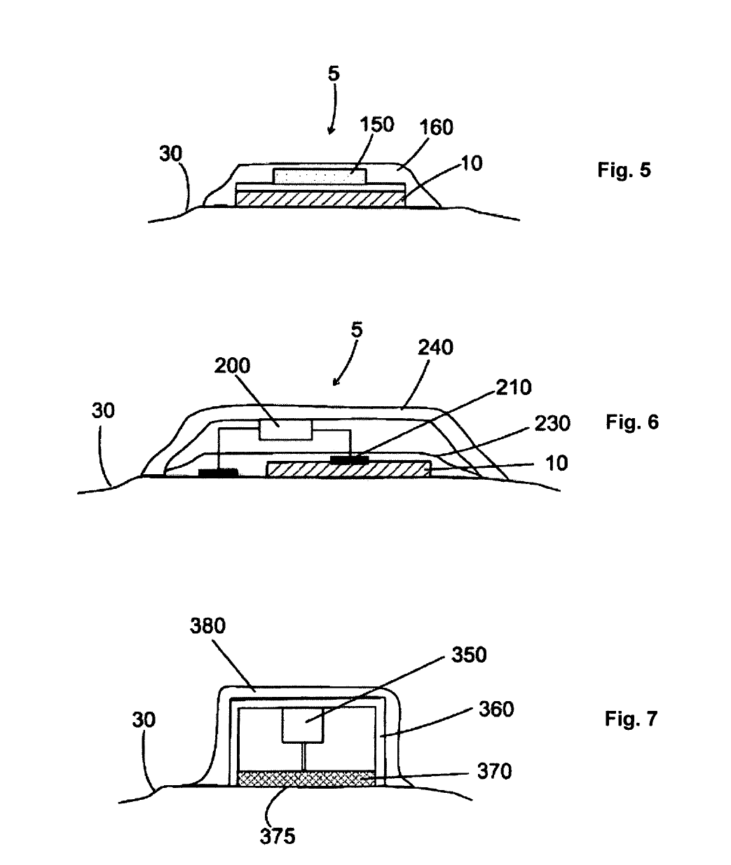

FIG. 5 shows a type of patch incorporating an rupturable liquid reservoir 150, surrounded by a malleable cover 160. The user can rupture the reservoir by pressing on the malleable cover, thus releasing a solvent, such as water, which then interacts with the dissolvable layer 10 thus enhancing dissolution of the dissovable layer.

FIG. 6 shows a patch incorporating an ionotophoresis unit. An electrode 210 is in contact with the dissolving layer 10, which is in contact with the skin 30. Another electrode 220 is in contact with the skin. A power supply and control electronics is contained in 200. A non-conducting layer 230 serves to hold the dissolvable layer and electrodes in place. Once dissolution of layer 10 begins, the iontophoresis process will begin and will enhance the delivery of the active material from the dissolving layer 10.

FIG. 7 shows a patch incorporating a method to ablate or reduce the stratum corneum on the skin 30. For example, a dissolving abrasive member 370 incorporating a dissolving layer coated with abrasive, such as aluminum oxide, or incorporating a rough textured surface, in contact with the skin 375. This abrasive member is driven to rotate, oscillate, or be drawn across the skin manually or by a motor and controller 350. A housing 360 incorporates the abrasive member and motor and controller. After the skin is abraded, the active materials released from the dissolving layer 370.

### Cover or Backing

This part of the patch may be transparent, opaque or even decorative. It should be thin and flexible, with a MWTR such that the skin to which it touches can “breathe” thus enhancing comfort and patient compliance. The cover may have a bar-code on the superior surface, or a radiofrequency identification tag (RFID) incorporated so that health-care-providers can easily keep track of the patch and patient to which it is applied. An liquid-crystal thermometer (reversible or non-reversible) may optionally be part of the cover since it is known what elevated temperatures can lead to a dangerously increased uptake of the active material from a transdermal patch; the thermometer would allow the temperature of the skin surface to be monitored thus warning the patient that a critical high temperature has been reached and the patient must seek a cooler environment. An non-reversible light-sensitive dye, e.g. cyanines or phthalocyanines, in the cover would provide a rough visual indication of wear time; comparison of the color change to a standard color scale would remind the patient that the patch has been applied for a critical amount of time.

Optionally, the cover may incorporate a material that heats the patch, thereby enhancing the dissolution of the dissolving layer using, for example, calcium chloride, carnallite, activated carbon, vermiculite, sodium acetate, sodium hydroxide, mixed with water when ready, lighter-fluid and platinum catalyst, or iron filings exposed to air, or cools the patch using, for example, ammonium nitrate and water mixed when cooling is required, thereby slowing down dissolution. Alternatively, a high-frequency external magentic field, radiofrequency surgical-type electrical currents, ultrasound, or light, could be directed onto the patch thereby inducing a rise in temperature.

### Membrane

Optionally, a membrane layer may be designed to allow for release of the active material, upon activation, whereby the activation consists of compromising the integrity of the membrane. Examples include perforation, or dissolution upon exposure to fluids or heat, e.g. body heat or heat produced by an endothermic chemical reaction, thus allowing the active agent to flow past the barrier. Alternatively, the adhesive layer may be separate from the membrane layer and may be porous or permeable whereby, upon mechanically compromising the integrity of a reservoir contained within the barrier layer or laminate, a bioactive material, such as a pharmaceutical, is released to diffuse through the adhesive layer.

### Dissolving Layer

The active material is incorporated into a dissolving layer, which may optionally additionally incorporate an adhesive and/or membrane. The dissolving layer compromises at least one ingredient that is excipients, surfactants, stabilizing agents, emulsifiers, thickeners, plasticizers, antimicrobials, water, water soluble polymers, binders, polyethylene oxides, propylene glycols, sweeteners, flavor enhancers, colorants, polyalcohols, and combinations thereof; and xanthones derived from a mixture of pulp and pericarp of fruit of Garcinia mangostana L. plant.

### Excipients

The dissolving film layer may optionally comprise in part or in whole a hydrocolloid. Preferably, the hydrocolloid comprises a water soluble natural polysaccharide or derivatives including pectin and derivatives, guar gum arabic, tragacanth gum, xanthan gum, gellan sodium salt, propyleneglycol alginate, starches (amylose, amylopectin), modified starches, hydroxyethyl starch, pullulan, carboxymethyl starch, gum ghatti, okra gum, karaya gum, dextrans, dextrins and maltodextrins, konjac, acemannan from aloe, locust bean gum, tara gum, quince seed gum, fenugreek seed gum, scleroglucan, gum arabic, psyllium seed gum, tamarind gum, oat gum, quince seed gum, carrageenans, scleraglucan, succinoglucan, larch arabinogalactan, flaxseed gum, chondroitin sulfates, hyaluronic acid, curdlan, chitosan, deacetylated konjac, and rhizobium gum.

The hydrocolloid may be a water soluble non-gelling polypeptide or protein exemplified by gelatins, albumins, milk proteins, soy protein, and whey proteins. The hydrocolloid further may be selected from a group of synthetic hydrocolloids exemplified by polyethylene-imine, hydroxyethyl cellulose, sodium carboxymethyl cellulose, carboxymethyl cellulose, hydroxypropyl cellulose, hydroxypropyl methyl cellulose, methyl cellulose, ethyl cellulose, polyacrylic acids, low molecular weight polyacrylamides and their sodium salts (carbomers), polyvinylpyrollidone, polyethylene glycols, polyethylene oxides, polyvinyl alcohols, pluronics, tetronics, and other block co-polymers, carboxyvinyl polymers, and colloidal silicon dioxide.

Suitable hydrocolloids or mixtures producing synergistic properties comprise natural seaweeds, natural seed gums, natural plant exudates, natural fruit extracts, biosynthetic gums, gelatines, biosynthetic processed starch or cellulosic materials, alginates, agar gum, guar gum, locust bean gum (carob), carrageenan, tara gum, gum arabic, ghatti gum, Khaya grandifolia gum, tragacanth gum, karaya gum, pectin, arabian (araban), xanthan, gellan, starch, Konjac mannan, galactomannan, funoran, are xanthan, acetan, gellan, welan, rhamsan, furcelleran, succinoglycan, scleroglycan, schizophyllan, tamarind gum, curdlan, pullulan, and dextran

Additionally, the dissolving layer may comprise any or all of emulsifying agents, solubilizing agents, wetting agents, taste modifying agents, plasticizers, active agents, water soluble inert fillers, preservatives, buffering agents, coloring agents, and stabilizers. Addition of a plasticizer to the formulation can improve flexibility. The plasticizer or mixture of plasticizers may be polyethylene glycol, glycerol, sorbitol, sucrose, corn syrup, fructose, dioctyl-sodium sulfosuccinate, triethyl citrate, tributyl citrate, 1,2-propylenglycol, mono-, di- or triacetates of glycerol, or natural gums. Preferred plasticizers are glycerol, polyethylene glycol, propylene glycol, citrates and their combinations. The amount of plasticizer depends on the final application.

Examples of natural water-soluble polymer include plant-type polymer, microorganism-type polymers and animal-type polymers. A plant-type polymer may be gum arabic, gum tragacanth, galactan, guar gum, carob gum, karaya gum, carrageenan, pectin, agar, quince seed or Cydonia oblonga, algae colloids such as brown algae extract, starches such as rice, corn, potato, and wheat, and glycyrrhizic acid. Microorganism-type polymers may be xanthan gum, dextran, succinoglucan, and pullulan. Animal-type polymers may be collagen, casein, albumin, and gelatin.

The water soluble polymer also may be selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

The film-forming agent used in the films according to the present invention may be selected from pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A preferred film former is pullulan, in amounts ranging from about 0.01 to about 99 wt %, preferably about 30 to about 80 wt %, more preferably from about 45 to about 70 wt % of the film and even more preferably from about 60 to about 65 wt % of the film.

Examples of the semisynthetic water-soluble polymers include starch-type polymers, cellulosic polymers and alginic acid-type polymers. Starch-type polymers may be carboxymethyl starch and methylhydroxypropyl starch. Cellulosic polymers may be methyl cellulose, ethyl cellulose, methylhydroxypropyl cellulose, hydroxyethyl cellulose, cellulose sodium sulfate, hydroxypropyl cellulose, carboxymetyl-cellulose, sodium carboxymethyl cellulose, crystal cellulose, and cellulose powder. Alginic acid-type polymers may be sodium alginate and propyleneglycol-alginate.

Examples of the synthetic water-soluble polymers include vinyl polymers, polyoxyethylene-type polymers, acrylic polymers, and cationic polymers, and polyethyleneimine. Vinyl polymers may be polyvinyl alcohol, polyvinyl methyl ether, polyvinylpyrrolidone, carboxy vinyl polymer. Polyoxyethylene-type polymers may be a copolymer of polyethylene glycol 20,000, 40,000, or 60,000 and polyoxyethylene polyoxypropylene. Acrylic polymers may be sodium polyacrylate, polyethylacrylate, and polyacrylamide.

Thickeners may include gum arabic, carrageenan, karaya gum, gum tragacanth, carob gum, quince seed or Cydonia oblonga, casein, dextrin, gelatin, sodium pectate, sodium alginate, methyl cellulose, ethyl cellulose, CMC, hydroxy ethyl cellulose, hydroxypropyl cellulose, PVA, PVM, PVP, sodium polyacrylate, carboxy vinyl polymer, locust bean gum, guar gum, tamarind gum, cellulose dialkyl dimethylammonium sulfate, xanthan gum, aluminum magnesium silicate, bentonite, hectorite, AIMg silicate or beagum, laponite, and silicic acid anhydride. Preferred thickening agents include methylcellulose, carboxyl methylcellulose, and the like, in amounts ranging from about 0 to about 20 wt %, preferably about 0.01 to about 5 wt %.

Preferred surfactants include mono and diglycerides of fatty acids and polyoxyethylene sorbitol esters, such as, Atmos 300 and Polysorbate 80. The surfactant can be added in amounts ranging from about 0.5 to about 15 wt %, preferably about 1 to about 5 wt % of the film. Other suitable surfactants include pluronic acid, sodium lauryl sulfate, and the like.

Preferred stabilizing agents include xanthan gum, locust bean gum and carrageenan, in amounts ranging from about 0 to about 10 wt %, preferably about 0.1 to about 2 wt % of the film. Other suitable stabilizing agents include guar gum and the like. A number of naturally occurring small organic molecules display chaperone-like activity, stabilizing the native conformation of proteins. Most of them are sugars, polyols, amino acids or methylamines. For example, the capacity of trehalose and glycerol, to stabilize and renature cellular proteins is well known.

Preferred emulsifying agents include triethanolamine stearate, quaternary ammonium compounds, acacia, gelatin, lecithin, bentonite, veegum, and the like, in amounts ranging from about 0 to about 5 wt %, preferably about 0.01 to about 0.7 wt % of the film. Preferred binding agents include starch, in amounts ranging from about 0 to about 10 wt %, preferably about 0.01 to about 2 wt % of the film. It may be necessary to additionally incorporate compounds that act as preservatives or buffers. An example of such a material is sodium benzoate.

### Active Agent

The expression “pharmaceutically active agents”, “active agent”, “active material” or “pharmaceutical” as used herein is intended to encompass agents other than foods, which promote a structural and/or functional change in and/or on bodies to which they have been administered. They may also be an agent used in the recognition of a disease or condition. These agents are not particularly limited; however, they should be physiologically acceptable and compatible with the film.

The dissolving film may be supplemented with at least one composition selected from, for example, one or more regulatory compounds, antibody, antimicrobial compositions, analgesics, anticoagulants, antiproliferatives, anti-inflammatory compounds, cytokines, cytotoxins, drugs, growth factors, interferons, hormones, lipids, demineralized bone or bone morphogenetic proteins, cartilage inducing factors, oligonucleotides polymers, polysaccharides, polypeptides, protease inhibitors, vasoconstrictors or vasodilators, vitamins, minerals, stabilizers and the like wherein said pharmaceutical agent is selected from the group consisting of a peptide, a hormone, a nucleic acid, a gene construct, an antigen, an adjuvant, an antibiotic, an anti-viral agent, an analgesic or analgesic combination, a local or general anaesthetic, and an anti-inflammatory.

The transport processes associated with this invention lend themselves to use with a wide variety of molecules including drugs and molecules of diagnostic interest. Molecules, e.g., active agents, which may be delivered by the method and/or device of the present invention include, but are not limited to, any material capable of exerting a biological effect on a human body, such as therapeutic drugs, including, but not limited to, organic and macromolecular compounds such as polypeptides, proteins, polysaccharides, nucleic acid materials comprising DNA, and nutrients. Examples of polysaccharide, polypeptide and protein active agents include, but are not limited to, heparin and fragmented (low molecular weight) heparin, thyrotropin-releasing hormone (TRH), vasopressin, gonadotropin-releasing hormone (GnRH or LHRH), melanotropin-stimulating hormone (MSH), calcitonin, growth hormone releasing factor (GRF), insulin, erythroietin (EPO), interferon alpha, interferon beta, oxytocin, captopril, bradykinin, atriopeptin, cholecystokinin, endorphins, nerve growth factor, melanocyte inhibitor-I, gastrin antagonist, somatostatin, encephalins, cyclosporin and its derivatives (e.g., biologically active fragments or analogs).

Other examples of active agents include anesthetics, analgesics, drugs for psychiatric disorders, epilepsies, migraine, stopping drug additions and buses; anti-inflammatory agents, drugs to treat hypertension, cardiovascular diseases, gastric acidity and GI ulcers; drugs for hormone replacement therapies and contraceptives; antibiotics and other antimicrobial agents; antineoplastic agents, immunosuppressive agents and immunostimulants; and drugs acting on blood and the blood forming organs including hematopoietic agents and anticoagulants, thrombolytics, and antiplatelet drugs. Other active agents suitable for transdermal delivery to treat allergies are selected from the group consisting of fine particles or extracts from natural substances, e.g., from herbs, grass seeds, pollens, and animal debris. Also, other cationic and anionic active agents, such as those described in M. Roberts, et al., “Solute Structure as a Determinant of lontophoretic Transport”, Mechanisms of Transdermal Drug Delivery, R. O. Potts and R. H. Guy, Ed., Marcel Dekker, pages 291-349, 1997, may be delivered with film-based systems described herein and in combination with a device utilizing iontophoresis or

Other suitable pharmaceutically active agents include, but are not limited to 1) antimicrobial agents, such as triclosan, cetyl pyridium chloride, domiphen bromide, quaternary ammonium salts, zinc compounds, sanguinarine, fluorides, alexidine, octonidine, EDTA, and the like; 2) non-steroidal anti-inflammatory drugs, such as aspirin, acetaminophen, ibuprofen, ketoprofen, diflunisal, fenoprofen calcium, naproxen, tolmetin sodium, indomethacin, and the like; 3) anti-tussives, such as benzonatate, caramiphen edisylate, menthol, dextromethorphan hydrobromide, chlophedianol hydrochloride, and the like; 4) decongestants, such as pseudoephedrine hydrochloride, phenylepherine, phenylpropanolamine, pseudoephedrine sulfate, and the like; 5) anti-histamines, such as brompheniramine maleate, chlorpheniramine maleate, carbinoxamine maleate, clemastine fumarate, dexchlorpheniramine maleate, diphenhydramine hydrochloride, diphenylpyraline hydrochloride, azatadine meleate, diphenhydramine citrate, doxylamine succinate, promethazine hydrochloride, pyrilamine maleate, tripelennamine citrate, triprolidine hydrochloride, acrivastine, loratadine, brompheniramine, dexbrompheniramine, and the like; 6) expectorants, such as guaifenesin, ipecac, potassium iodide, terpin hydrate, and the like; 7) anti-diarrheals, such a loperamide, and the like; 8) H₂-antagonists, such as famotidine, ranitidine, and the like; 9) proton pump inhibitors, such as omeprazole, lansoprazole, and the like; 10) general nonselective CNS depressants, such as aliphatic alcohols, barbiturates and the like; 11) general nonselective CNS stimulants such as caffeine, nicotine, strychnine, picrotoxin, pentylenetetrazol and the like; 12) drugs that selectively modify CNS function, such as phenyhydantoin, phenobarbital, primidone, carbamazepine, ethosuximide, methsuximide, phensuximide, trimethadione, diazepam, benzodiazepines, phenacemide, pheneturide, acetazolamide, sulthiame, bromide, and the like; 13) antiparkinsonism drugs such as levodopa, amantadine and the like; 14) narcotic-analgesics such as morphine, heroin, hydromorphone, metopon, oxymorphone, levorphanol, codeine, hydrocodone, xycodone, nalorphine, naloxone, naltrexone and the like; 15) analgesic-antipyretics such as salycilates, phenylbutazone, indomethacin, phenacetin and the like; and 16) psychopharmacological drugs such as chlorpromazine, methotrimeprazine, haloperidol, clozapine, reserpine, imipramine, tranylcypromine, phenelzine, lithium and the like.

The amount of pharmaceutically active agent that can be used in the rapidly dissolving films, according to the present invention, is dependent upon the dose needed to provide an effective amount of the pharmaceutically active agent.

Similarly, biomolecules and other substances of diagnostic interest, including both naturally occurring substances and therapeutically introduced molecules in interstitial fluid may be collected in these films for subsequent assaying. In this instance the film preferably does not completely dissolve, and absorbs the analyte. These molecules and substances include, but are not limited to, natural and therapeutically introduced metabolites, hormones, amino acids, peptides and proteins, polynucleotides, cells, electrolytes, metal ions, suspected drugs of abuse, enzymes, tranquilizers, anesthetics, analgesics, anti-inflammatory agents, immunosuppressants, antimicrobials, muscle relaxants, sedatives, antipsychotic agents, antidepressants, antianxiety agents, small drug molecules, and the like. Non-limiting representative examples of such materials include glucose, cholesterol, high density lipoproteins, low density lipoproteins, triglycerides, diglycerides, monoglycerides, bone alkaline phosphoatase (BAP), prostate-Specific-Antigen (PSA), antigens, lactic acid, pyruvic acid, alcohols, fatty acids, glycols, thyroxine, estrogen, testosterone, progesterone, theobromine, galactose, uric acid, alpha amylase, choline, L-lysine, sodium, potassium, copper, iron, magnesium, calcium, zinc, citrate, morphine, morphine sulfate, heroin, insulin, interferons, erytheopoietin, fentanyl, cisapride, risperidone, infliximab, heparin, steroids, neomycin, nitrofurazone, betamethasone, clonidine, acetic acid, alkaloids, acetaminophen, and amino acids. In one embodiment, more than one substance is sampled at one time.

### Vaccines

A special type of active agent is a vaccine, which normally is not intended to treat disease, although there are some vaccines that can, but to prevent disease by improving immunity to the antigen that effects the disease. Vaccines can be made up of various antigens such as killed microorganisms, live but attenuated viruses, toxoids, fragments of the infectious micro-organism (subunit vaccine), the polysaccharide outer coat of certain bacteria of viruses (conjugate), recombinent vectors or DNA. Any of these vaccines can be incorporated into the dissolving layer, although consideration must be taken of the stability of the antigen. It is hypothesized that the stability of antigens incorporated into the dissolving layer are enhanced because of the solid or semi-solid nature of the layer which serves to keep the antigens from self-associating.

Some of the most important vaccines developed or currently being studied for intradermal vaccination are for smallpox, tuberculosis, yellow fever, rabies, hepatitis B, influenza, polio, cholera, measles, typhoid, tetanus, hepatitis A, traveller's diarrhea. All of these vaccines and vaccine-candidates, and others not mentioned, would be beneficially delivered transdermally using the present invention.

### Adjuvants

A pharmacologic adjuvant is a drug that increases the efficacy or potency of other drugs when given at the same time; for example, caffeine administered with acetaminophen has an analgesic effect better than each drug alone. An immunological adjuvant is an agent that stimulates the immune system, and so increases the immunogenicity of a vaccine. Examples of adjuvants are alum, squalene, saponins, virosomes, or oil-based adjuvants. All of these adjuvants can optionally be incorporated into the dissolving layer of the patch.

Other non-traditional adjuvants may work to enhance dendritic cell (DC) cell migration, activate toll-like receptors, activate T-cells, upregulate production of B and T cells, stimulate chemokine releasing helper T cells and mast cells, or induce release of inflammatory cytokines. For example, DC migration is regulated by cytokines such as TNF-alpha and IL-β and to involve adhesion molecules such as I-CAM, E-caherin, ingtegrin-α6 and CD44. DCs create a path to the draining lymph node by digesting collagen in connective tissue, basement membranes and dermal extracellular matrix by metalloproteases 2 and 9 [32]. Materials, such as osteonectin, may enhance the ability of DC's to migrate and so may act to vaccine immunogenicity. Other nono-traditional adjuvants are toll-like-receptor agonists, CpG motifs, all-trans retinoic acid, heat-labile toxin, and cholera toxin. Such non-traditional adjuvants may be beneficially incorporated into the dissolving layer. An interesting family of photoimmunomodulators, such as psoralens or porphyrins, may also be efficiently delivered using this dissolving layer patch.

Adjuvants can be a problem in that they complicate the dissolving layer formulation, or induce side-effects. The present invention does not necessarily require adjuvant when vaccine is delivered, and in fact experimentation shows that with some vaccines, immunogenicity equivalent to intramuscular injection results when the patch invention is used to trancutaneously administer vaccine; this is an unexpected and surprising observation, and goes against current dogma.

### Additional Active Components

The dissolving layer of the patch requires water, or other liquid solvents, to dissolve. Materials that enhance or decrease moisture production and release from the body may serve to enhance or modulate the dissolution. For example, it may be beneficial to incorporate into the dissolving layer components that increase sweating, e.g. pilocarpine, cortisone, or reduce sweating, e.g., aluminum chloride, botulinum toxin A, other anticholinergic drugs such as oxybutynin, glycopyrrolate, benztropine or propantheline bromide. Alternatively, materials that enhance dissolution of the dissolving layer could be used beneficially; for example, pullulanase could be used to enhance the breakdown of pullulan in the dissolving layer thus increasing the speed at which the active material partitions out of the patch.

Altering the behavior of the skin, in terms of it's barrier function, can also be beneficial. For example, after the stratum corneum is altered such that the barrier function is reduced, certain drugs can be incorporated into the dissolving layer to inhibit or enhance regeneration of the barrier so that the permeation of the active material continues for an extended time or is quickly inhibited; an example of such drugs are antimetabolites or capsaicin, or enhance barrier recover (hydrocortisone).

Permeation enhancers, e.g. water, azone, alcohol, dimethyl-sulfoxide, have been shown to work on intact skin, that is skin with intact SC, but the present invention raises the need for a new generation of permeation enhancers. The alteration or ablation of the SC exposes a cellular milieu with chemical properties very different from the SC, and so permeation enhancers that work on the exposed dermis, e.g. hydrophilic, instead of the intact stratum corneum, which is hydrophobic, could be beneficially incorporated in the dissolving layer of the patch.

Skin flora are the microorganisms which reside on the skin, mostly bacteria, and often provide protection to the body by preventing pathogenic organisms from colonizing on the skin surface. These materials, along with proteases and other enzymes within the skin can be a problem for topically applied materials, such as vaccines or protein-based drugs, for example. Incorporating materials into the dissolving layer that serve to inhibit these microorganisms, e.g. antibiotics, or enzymes, e.g. serine protease or peptidase inhibitors such as neuroserpin or lipocalin proteins, could be beneficial.

### Physical Properties of Dissolving Layer

By choosing the physical properties of the dissolving layer, it is possible to control the delivery of the active material to the tissue. For example, the size (area, cm²) of the dissolving layer in contact with the tissue determines the dose rate (mg/hr) and the total amount (mg) of active material delivered. The flux (mg/hr/cm²) is a property that is important to consider; for example, particular active materials are toxic to the tissue at critical dose intensities (gm/cm²). To reduce local toxicity, and to increase dose rate, it may be beneficial to increase the area of the dissolving layer that is in contact with the tissue.

A thicker dissolving layer, or a dissolving layer formulated with certain excipients (e.g. hydroxypropylcellulose) which inhibit dissolution, can be used to control the rate at which the active material is delivered from the patch.

### Adhesive Shield

Additionally, the present invention provides a shield or laminate for an adhesive or an adhesive laminate whereby an external layer of the shield may dissolve upon contact with fluids. The soluble, external shield may serve as an applicator to facilitate the application of the adhesive to tissue. The shield or laminate also may comprise one or more internal layers, reservoirs or pooled materials containing the adhesive. Furthermore multiple layers or reservoirs may contain a biologic, a drug or other pharmaceutical substance, whereby the system becomes a drug delivery device. The bioactive material may be, although not limited to, one of or a combination of nitroglycerin, an anti-nauseant, an antibiotic, a hormone, a steroidal anti-inflammatory agent, a non-steroid antiinflammatory agent, a chemotherapeutic agent, an anti-cancer agent, an immunogen, an analgesic, an anti-viral agent or an anti-fungal agent.

The layers used as a barrier to separate the adhesive from the backing, or the adhesive from the substrate, may be removed upon application. Alternatively, a layer may be designed to allow for release of the active agent, e.g., adhesive or biologic, drug or other pharmaceutical substance, upon activation, whereby the activation consists of compromising the layer. Examples include perforation, or dissolution upon exposure to fluids or heat, e.g. body heat, thus allowing the active agent to flow past the barrier. Alternatively, the adhesive layer may be separate from the barrier layer and may be porous or permeable whereby, upon mechanically compromising the integrity of a reservoir contained within the barrier layer or laminate, a bioactive material, such as a pharmaceutical, is released to diffuse through the adhesive layer.

### Further Embodiments

The invention provides a means for monitoring of the levels of glucose or glucose metabolite, e.g., lactic acid, from the body. The method can also be used to collect fluids into a film layer for measurement of blood substance (glucose) levels in either a semi-continuous or a single measurement method. The method can be practiced further by a device that provides electrodes or other means for applying electric current to the tissue at the collection site; one or more collection reservoirs or sampling chambers to receive the substance (glucose); and a substance concentration measurement system. U.S. Pat. Nos. 5,735,273, 5,827,183, 5,771,890 describe the method of reverse iontophoresis for non-invasive interstitial fluid sampling for diagnostic purpose.

The drug-delivery patch can have additional drug-delivery devices incorporated within the patch. For example, optionally an abrasive system (FIG. 6) whereby the stratum corneum is compromised by abrasion, such as would result from drawing an member coated with abrasive such as aluminum oxide across the skin, could be used prior to application of the active agent. Alternatively, an ionotophoretic system could be engaged after the application of the drug-delivery patch (FIG. 5) could be used to enhance the delivery of the active agent from the patch to the tissue to be treated.

In some cases, it would be beneficial to incorporate a biocompatible dye, e.g. methylene blue or indocyanine green, into the dissolving layer so that when the patch is removed, evidence of dissolution will remain on the surface of the tissue where the patch was applied.

In all aspects of this embodiment the excipient dissolves upon contact with a fluid, e.g., water or interstitial fluid. The excipient may be a hydrocolloid such as pullulan. One or more layers of the biocompatible excipient substance may comprise further an emulsifying agent, a solubilizing agent, a wetting agent, a taste modifying agent, a plasticizer, an active agent, a water soluble inert filler, a preservative, a buffering agent, a coloring agent, an aesthetic design, a stabilizer, or a combination thereof.

Optionally, in order to increase the rate of dissolution, it would prove beneficial to apply a layer of moist material over the dissolvable layer after the patch is applied to the skin. This layer, which can consist of, for example, a hydrogel, could be occluded with the cover or backing in order to contain the moisture against the dissolving layer and skin. If used, this moist material layer may be beneficially packaged separately from the dissolving patch layer and applied by the patient over the dissolving layer when required.

### Methods of Manufacture

One way to manufacture the solid dissolving layer is to pour the (liquid pre-form) layer formulation onto a non-stick substrate, e.g. polytetrafluoroethylene, or PTFE, which can be inclined to induce thinning of the material as it drys. Alternatively a draw-down technique can be used whereby a narrow aperture is drawn across the liquid thus spreading it out into a known thickness. Depending on viscosity, angle of incline, nature of substrate and ambient temperature and humidity, layers of different thickness will result.

Another way to manufacture the dissolving layer is to use a blown film extrusion process similar to the way that plastic films are made. In this case, an extruding cylinder of liquid pre-form is continuously inflating to several times its initial diameter, thus forming a thin tubular films which can be cut and shaped. Additional details or steps to this process, such as blowing in chilled air to enhance solidification process, or altering the pull-off speed, can result in a dissolving layer with unique and controllable properties.

Here, an excess amount of pre-form liquid is placed on a rotating substrate, e.g. PTFE, often a drum, and allowed to spread by centrifugal force. The thickness of the resulting solid film depends on the rotation speed, ambient temperature and humidity, rheology of the pre-form liquid, and the concentration of the excipient and solvent.

In spray forming or casting, the pre-form liquid is changes into an aerosol mist of liquid particles by exciting from a container than contains the liquid under pressure. The aerosol mist is directed as a substrate, such as PTFE, at a beneficial temperature and in a dry atmosphere, so that the particles quickly dry on the substrate surface. Layers of controllable thicknesses, or with different active materials, can be superimposed thus providing a dissolvable layer with preditable drug-delivery properties.

The following examples are given for the purpose of illustrating various embodiments of the invention and are not meant to limit the present invention in any fashion.

### EXAMPLE 1

### Formulation of the Dissolving-Layer Patch

The dissolvable layer of the drug-delivery patch, is largely a binary formulation consisting of a water-soluble polysaccharide polymer and water, with a small amount of a plasticizer and surfactant. Antigen is added to the patch material when it is in liquid form, and the viscous mixture (monitored by a Brookfield viscometer for quality control) is poured onto a polytetrafluoroethylene plate while drying. The surfactant aids materials dispersion for consistent drawdown during casting. A polyurethane backing is applied to the outer surface of the film. The patch is convenient for dosing, suitable for labeling, and flexible for easy packing, handling and application. The thickness of a typical film ranges from 10-160 μm, and its surface area can be 1 to 20 cm² of any geometry. Its low dry-tack allows for ease of handling and application. At the same time, the rapid hydration rate (in the presence of moisture) facilitates an almost immediate softening of the dissolvable layer upon application to the reduced dermis or other moist issue. This layer has been applied to the skin of mice and human volunteers without any deleterious effects.

The active material is released from the dissolvable layer upon disintegration and dissolution. The disintegration and dissolving times were seen to be further influenced by varying the film thickness, or by varying the formulation composition of the film. The typical disintegration time, which is defined as the time at which the film begins to break when brought into contact with water, is only 5 to 10 seconds at thickness of 40 μm and the formulation described above.

The physical and mechanical properties of the dissolving layer are primarily controlled by the formulating and manufacturing process and are usually measured by in vitro testing methods: thickness, dry-tack, tensile strength, percent elongation, tear resistance, and Young's Modulus. Other performance properties, such as wet tack, bending length, disintegration time, dissolving time, and dissolution time, are conducted as quality control tests. Release of active ingredients may be modulated from minimal to >97% depending on formulation and available fluids.

Since the material dissolves in the presence of moisture, classic US Pharmacopoeia release tests for transdermal patches are irrelevant. Thus, in vivo release tests were designed using thin films impregnated with FD&C No. 1 dye, or ¹⁴C-lidocaine. In one test, the backs of BALB/c mice were treated in multiple sites with an abrasive device (FAST™ device, see, for example, United States Publication No. 20040236269, incorporated herein by reference) where a 60 μm abrasive, was applied to the skin with 10-20 grams of force, oscillating at 840 Hz for 2 seconds over a 5×8 mm spot. FD&C dye impregnated dissolvable films were placed over each treatment site with a semi-occlusive covering (patch). The patch was left on for a period of 1-24 hours. One hour after application, presence of dye on the mouse skin was visually noted, and estimated at >75%. A second, more quantitative release assay was performed using ¹⁴C-lidocaine impregnated in the patch. In these studies, after 24 hours, <3% of the lidocaine remained in the patch. The data are indicative that nearly all of the lidocaine was released from the patch and into the skin. This method and device therefore provides a means of delivering a metered dose of an active agent to the skin.

### EXAMPLE 2

### Other Dissolvable Layer Formulations

In all aspects of this invention, the film dissolves upon contact with a fluid, e.g., water or interstitial fluid that is released from the treatment site through compromise of the stratum corneum, which in turn releases the active agent into the tissue. The film may be comprised of a hydrocolloid such as pullulan. The film may be comprised of one or more layers, any of which may be comprised further of an emulsifying agent, a solubilizing agent, a wetting agent, a taste modifying agent, a plasticizer, an active agent, a water soluble inert filler, a preservative, a buffering agent, a coloring agent, an aesthetic design, a stabilizer, or a combination thereof.

Formulations for the dissolvable layer may include 1) fast-dissolving film component such as pullulan, generally 10-95% wt %; 2) a plasticizer for flexibility such as beta-carageenan, generally 0.05-35% wt %; 3) a dissolution modulating agent, e.g. hydroxymethycellulose, generally 0.1%-10%; and 4) a surfactant, for dispersion, such as polysorbate A at 0.001-0.1%. The initial preparation is mixed in deionized water and cast on a Teflon plate or releasable membrane, then allowed to dry. Thickness is determined by the composition of the formulation and completeness of the drawdown. Final residual water content is generally 1-4% depending on method of casting and extent of drying. An occlusive membrane, alumnized mylar, with an adhesive backing is typically applied applied over the patch.

In an example of a vaccine patch formulation the dissolvable layer is made up of 2.05% pullulan, 0.086% beta-carageenan, 0.014% polysorbate A and 160 ml of deionized water prior to addition of antigen and subsequent draw-down. The antigen concentration is such that 50 μg of antigen is in patch material with area of 5×8 mm and thickness of 30-160 μm. The film is cast on a Teflon plate or releasable membrane, allowed to dry. Thickness of the film is determined by composition, and is affected as well by final moisture content which is further affected by the extent of the drawdown.

### EXAMPLE 3

SC Ablation with FAST™ and TCI using Hemagluttinin or Recombinant Protective Antigen and Cellulose Type Patch

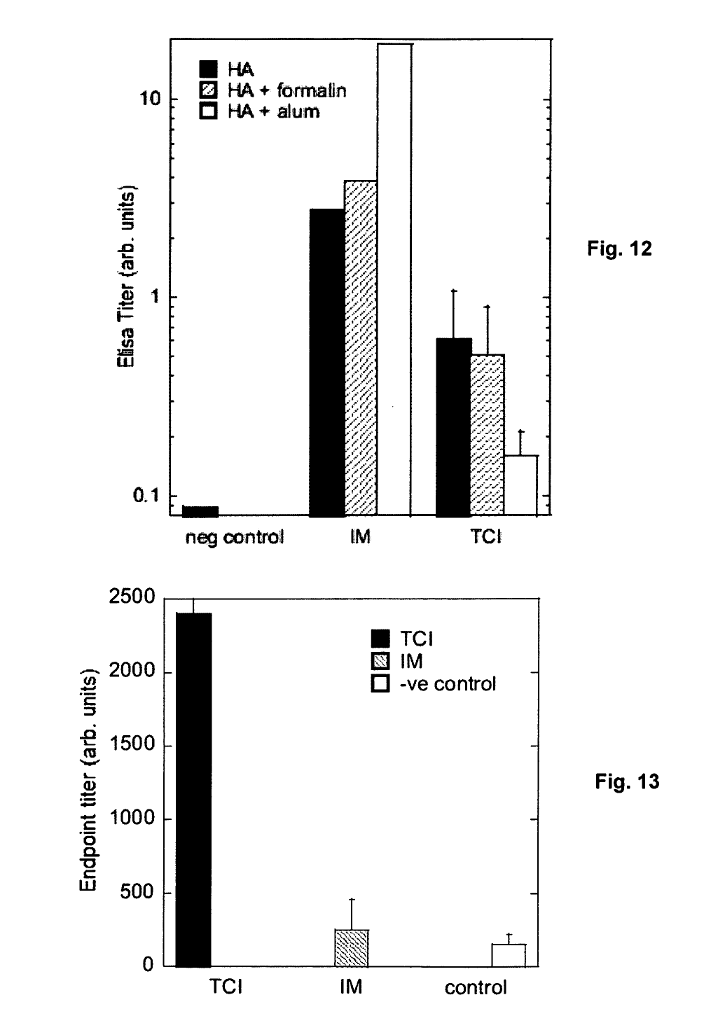

The results of a TCI experiment performed without the aid of a dissolvable layer patch is shown in FIG. 12. In this experiment, Influenza H5 Hemagluttinin (HA) or recombinant protective antigen (rPA) were used for immunization of mice following abrasive SC ablation (see United States Publication No. 20040236269). The device used in these experiments oscillates at 840 Hz, with skin-abrasive particles of 60-90 microns and applicator-skin pressures of 10-20 g.

In general, BALB/c or A/J mice (randomly male and female) were anesthetized, and the dorsal hair shaved and depilated. An approximate 5×8 mm spot was treated with the SC ablation device. Following treatment, an antigen (HA, 3 μg per dose or rPA, 10 μg per dose) incorporated into a patch made up of a 1×1 cm piece of cellulose tissue (e.g. Kimwipe®) covered by a semi-occlusive polyurethane dressing (e.g. 3M® Tegaderm®) for about 12 hours. In the case of rPA, adjuvants tested included γ-interferon (gINF) or lipopolysaccharide (LPS). Positive control mice received an intramuscular (IM) injection of vaccine, while negative control mice had the skin-treatment device treatment and distilled water applied in place of antigen. Antigen application occurred additionally at 14 days and 28 days, prior to sera collection. Sera were obtained pre-vaccination, and at 35 days post-vaccination. Sera were then analyzed by ELISA for the presence of reactive antibodies.

The results of this experiment, shown in FIG. 12, show that an immune response was generated with TCI, however IM injections were produced a higher titer antibody response than TCI. An adjuvant and preservative used in the study (formalin or alum) resulted in higher titer antibody response to the IM injections. The tested adjuvant seemed to reduce the efficiency of TCI as compared to the adjuvant free formulation, whereas formalin did not show a statistically significant difference.

### EXAMPLE 4

SC Ablation with FAST™ and TCI using Recombinent Protective Antigen and Dissolving-Layer Type Patch

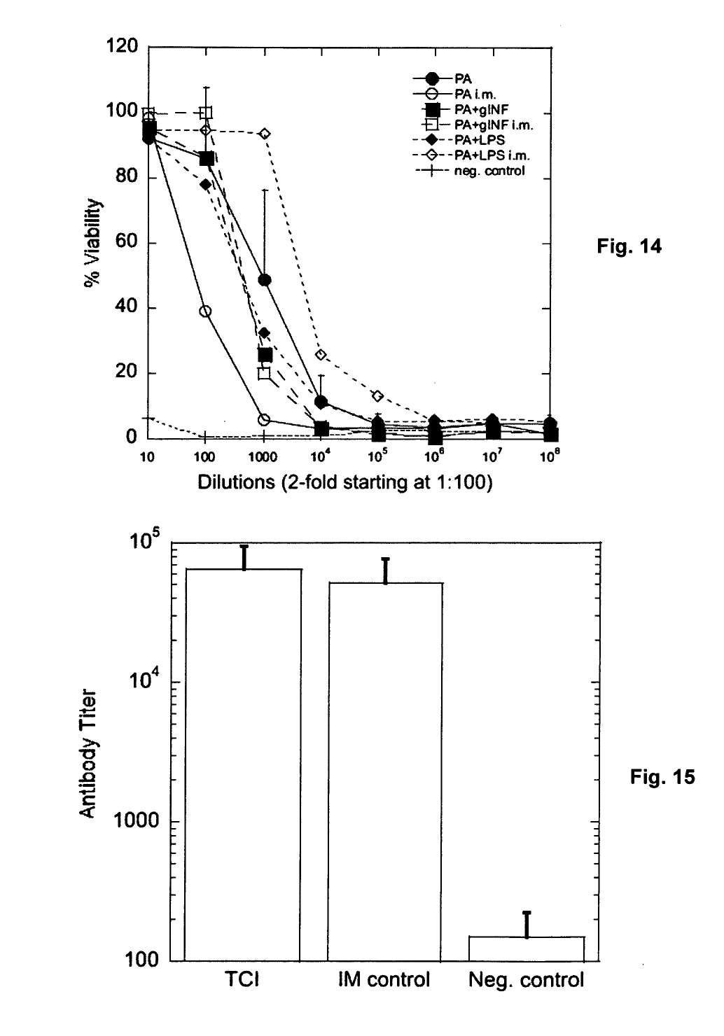

Experiments were performed using various antigens for immunization of mice following SC ablation with a SC ablation device. In these experiments, an antigen (recombinent anthrax protective antigen-rPA 10 μg per dose) was incorporated into a dissolving-layer patch. Antigens were added to a solution of 2.05% wt % pullulan, 0.086% wt % β-carageenan, 0.014% wt % polysorbate A and 160 ml of deionized water, poured onto a PTFE plate and air-dried at room temperature overnight. The patches were placed over the treated site in BALB/c mice, and covered by a semi-occlusive polyurethane dressing for about 12 hours. Positive control mice received an intramuscular (IM) injection of vaccine, while negative control mice had the skin-treatment and distilled water applied in place of antigen. In one experiment (FIG. 13) antigen application occurred additionally at 12 days and sera were collected at 15 days. In a second experiment, antigen was applied at 0, 14 and 28 days, prior to sera collection at 35 days. Sera were analyzed by ELISA for the presence of reactive antibodies. The results of the former experiment are shown in FIG. 13 and the latter experiment in FIG. 14. The ELISA assays demonstrated a significant response to rPA exceeding the IM controls. Brief immunization in the first experiment demonstrated greater titers of reactive IgG in transdermally vaccinated mice verses IM immunized mice (rPA).

In the second experiment, titers of anti-rPA IgG were not as great in TCI versus IM immunized mice (not shown). The rPA sera were further analyzed in an in vitro neutralization assay [19]. This assay measured cell survival after an in vitro challenge with anthrax toxin (LT) and, thus, is a more rigorous estimation of the ultimate in vivo effectiveness of TCI. A positive control used in the assay is a monoclonal antibody that binds to rPA with high efficiency and effectively neutralizes LT. The results of this assay are shown in FIG. 14. In this assay, sera from transcutaneous immunized mice had a high titer of neutralizing antibody. In many cases, the transcutaneously immunized mice had titers as high as the IM immunized controls. As the anti-PA titers of sera from cutaneously immunized mice were lower than IM controls, the results suggest that a greater fraction of immunoglobulin in the cutaneous mice was neutralizing.

### EXAMPLE 5

SC Ablation with FAST™ and TCI using Norwalk Virus Like Particles and Dissolving-Layer Type Patch

Experiments were performed using various antigens for immunization of mice following SC ablation with a SC ablation device. In this experiment, an antigen, Norwalk virus-like-particles (nVLP) at 5 μg per dose was incorporated into a dissolving-layer patch. Antigens were added to a solution of 2.05% wt % pullulan, 0.086% wt % β-carageenan, 0.014% wt % polysorbate A and 160 ml of deionized water, poured onto a PTFE plate and air-dried at room temperature overnight. The patches were placed over the treated site in BALB/c mice, and covered by a semi-occlusive polyurethane dressing for about 12 hours. Positive control mice received an intramuscular (IM) injection of vaccine, while negative control mice had the skin-treatment and distilled water applied in place of antigen. Antigen application occurred additionally at 14 days and 28 days, prior to sera collection. Sera were obtained pre-vaccination, and at 35 days post-vaccination. Sera were then analyzed by ELISA for the presence of reactive antibodies. The results of the experiment are shown in FIG. 15. The ELISA assay demonstrated a significant response to nVLP which was not significantly different from IM controls.

### EXAMPLE 6

### Dissolution of Patch Layer

The antigen is released from the dissolving layer upon disintegration and dissolution when in contact with fluids expressed from the site of SC ablation. The disintegration and dissolving times are influenced by varying the film thickness t, or by varying the dissolvable components formulation of the film. For example, in some experiments, film dissolution times varied from 0.5 minutes (t=30 μm) to 23.5 minutes (t=120 μm). By varying concentration of added hydroxypropylmethyl cellulose (HPMC; a cosmetic thickener and emulsifier) or hydroxymethylcellulose (HMC) in the formulation, it was possible to greatly extend the dissolution time. For example in experiments, a 250 μm patch prepared from a formulation of 0.125% w/w HPMC, 2.86% Pullulan, takes over 1.7 hours to dissolve. Thus, dissolution time can be modulated through the addition of HPMC.

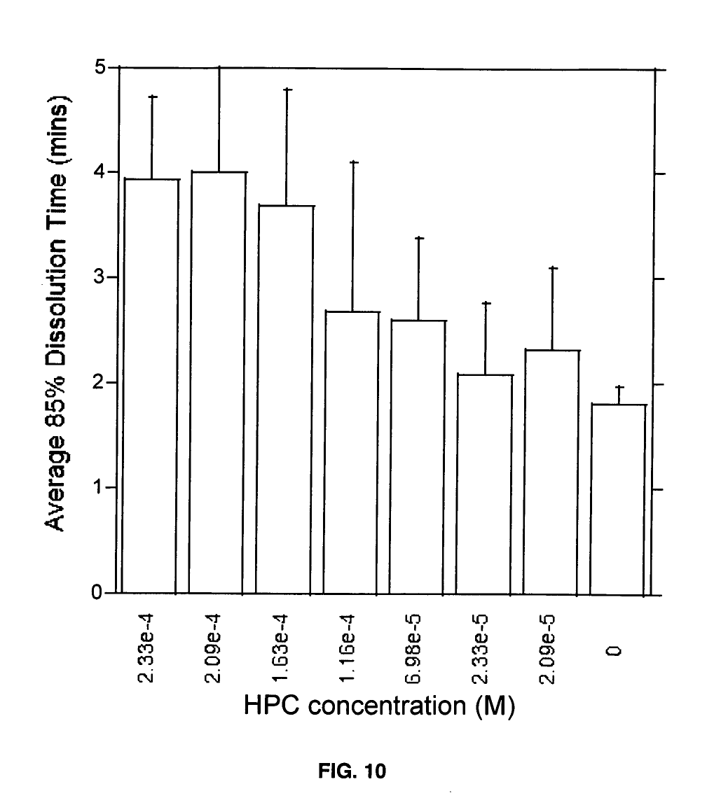

In another in vitro dissolution study, a 10×10 mm piece of the dissolving layer patch was cut from the middle of sample patches, weighed, and the average thickness measured. Each sample was then immersed in a beaker of 200 ml deionized water and 0.0005% polysorbate-80, adjusted to pH=5.0 (like stratum corneum) and held at a constant temperature of 37° C. and stirred at ˜200 rpm. At various times (5, 10, 15, 20, 30, 45, and 60 minutes) after patch immersion, 1 ml samples of water were taken and tested spectrophotometrically for optical absorbance at the peak of the trypan blue dye absorbance. The area of the absorption peak was calculated and compared to the total amount of dye mixed in the patch. These dissolution times were defined as the time that 85% of the dye was released from the sample patch (FIG. 10).

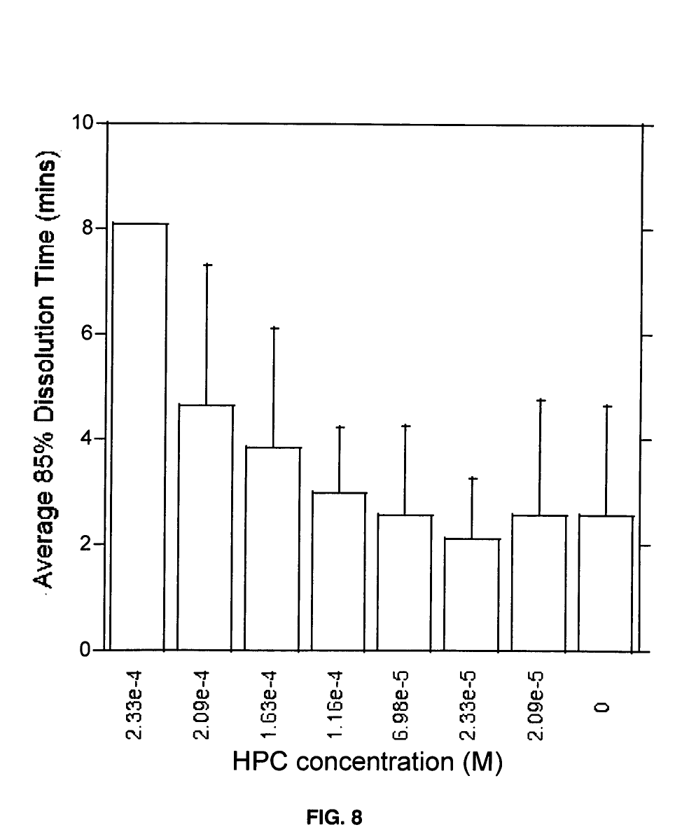

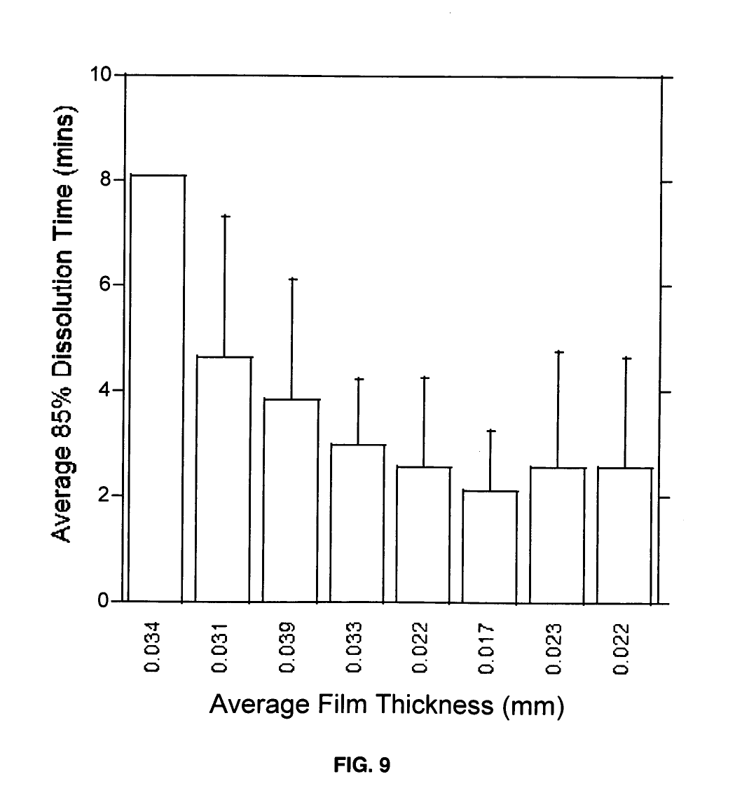

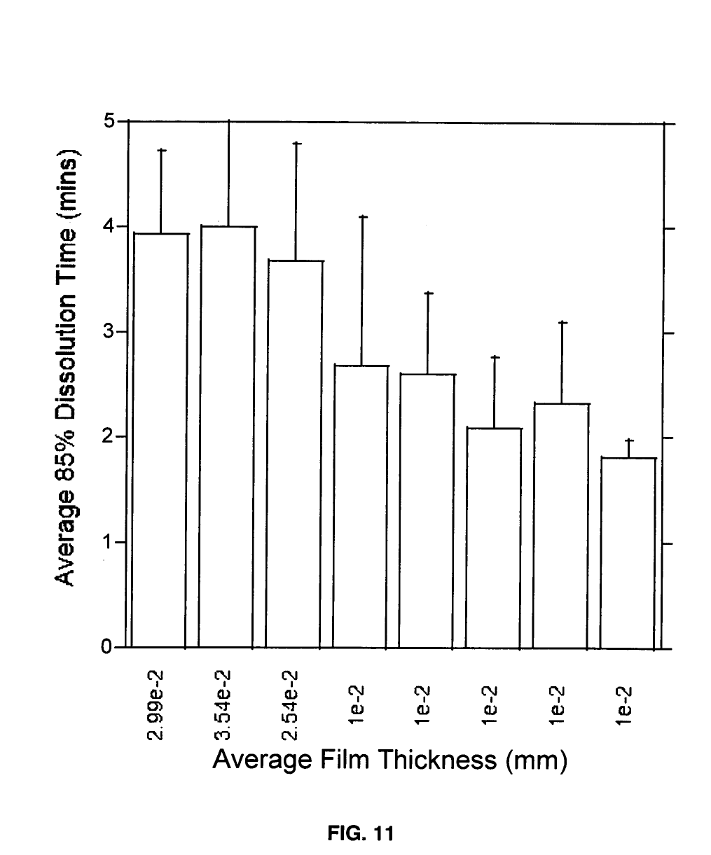

The results shown in FIGS. 8-11, for different dissolving layer thicknesses and concentrations of HPMC or HPC. Basically, the thicker the patch, the longer it takes to dissolve, and the more HPMC or HPC in the patch, the longer it takes to dissolve. The results suggest that both dosage and duration of exposure may be modulated using various formulations in the dissolvable layer of the patch. Since the kinetics of antigen exposure and dose effects to the skin have a significant effect on the immune response, the ultimate immune response may be modulated by adjusting formulations as discussed above.

Further modulation of the immune response may involve the delivery of antigens and immune response stimulators, or adjuvants, simultaneously, or at different times. For simultaneous administration, materials may be compounded together or in multiple layer patches. For timed release, multiple dissolvable layers may be used whereby each layer has a different dissolution rate. For example, the first layer may dissolve quickly, releasing an immune response modulator into the tissue. Such a modulator could act to activate dendritic cells or attract populations of cells through chemotaxis. A second layer could then release the antigen over a longer period of time, thus maximizing exposure to the activated cells, or larger population.

The following references are cited herein.
1. Zatz J. (ed.). Skin Permeation. Fundamentals and Applications. 1993. Allured Pub. Corp: Wheaton, Ill.
2. Shah, V. P. and Maibach, H. I. (eds.). Differences Between Oral and Transdermal Drug Delivery (G. W. Cleary, Transdermal Delivery Systems: A Medical Rationale in “Topical Drug Bioavailability, Bioequivalence and Penetration” pp. 17-68, Plenum Press, New York, 1993.
3. Cooper et al., New Eng. J. Med. 351, 22-29, 2004.
4. Belshe et al., New Eng. J. Med. 351, 2004.
5. Glenn et al., Nature 391:851.
6. Glenn et al., J. Immunol. 161:3211-3214, 1998.
7. Glenn et al., Infect. Immun. 67:1100-1106, 1999.
8. Hammond et al., Vaccine 19:2701-2707, 2001.
9. Shi et al., Vaccine 17, 2136-2141, 1999.
10. Cui et al., J. Control. Release 81, 173-184, 2002.
11. Hamilton J., J. Fam. Prac., August 1995.
12. Belyakov et al., J. Clin. Invest. 113:998-1007, 2004.
13. Metze et al., J. Invest. Dermatol. 96, 439-445, 1991.
14. Matyas et al., Infect. Immun. 72, 1181-1183, 2004.
15. Glenn, G. and Alving C., U.S. Pat. No. 6,797,276, Sep. 28, 2004.
16. Koh et al., Internet J. Anesth. 6(2), 2003.
17. Singh S., Drug Deliv. Report, Autumn/Winter 2005.
18. Godefroy et al., Infect. and Immun., 73:4803-4809, 2005.
19. Matyas et al., Infect. Immun. 72(2):1181-1183, 2004.
20. Marchitto, K. and Flock, S., U.S. Patent Publication No. 20060100567.
21. Glenn et al., Expr. Rev. Vaccines 2, 253-267, 2003.
22. Peachman et al., Infect. Immun. 74, 794-797, 2006.
23. Mikszta et al., JID 191, 278-288, 2005.
24. Proksch et al., British Journal of Dermatology, 134: 630-638, 1996.
25. Cumberbatch et al., Arch. Dermatol. Res. 288, 739-744, 1996.
26. Lutz et al., J. Immunol. 159, 3707, 1997.
27. Banchereau, J and Steinman, R. M., Nature 392, 245, 1998.
28. Lugar et al., J. Am Acad. Dermatol. 24, 915-926, 1991.
29. Lawley, T. J. and Kubota, Y., Semin. Dermatol. 10 256-259,1991.
30. Glenn et al., Immunol. Allergy Clin. North Am. 23:787-813, 2003.
31. Glenn, et al. Infection and Immunity, 75:5 p. 2163-2170.
32. Sangaletti S et al. J. Cell Sci. 118, 3685-3694, 2005

Any patents or publications mentioned in this specification are indicative of the levels of those skilled in the art to which the invention pertains. These patents and publications are herein incorporated by reference to the same extent as if it was indicated that each publication was incorporated specifically and individually by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A drug-delivery patch, comprising:
at least one dissolvable layer, wherein said layer comprising an active material; and
an adhesive backing or cover.

2. The patch of claim 1, further comprising a layer having a very low moisture vapor transmission rate.

3. The patch of claim 2, wherein said layer having a very low moisture vapor transmission rate comprises an aluminized polyester film, polyethylene or other plastic wrap.

4. The patch of claim 1, wherein said dissolvable layer comprises a first layer and a second, each layer having a different active material.

5. The patch of claim 4, wherein said first layer comprises an adjuvant and said second layer comprises an antigen.

6. The patch of claim 1, further comprising a second layer with a rupturable liquid reservoir surrounded by a malleable cover.

7. The patch of claim 1, further comprising an additional membrane layer that further modulates release of the active material.

8. The patch of claim 1, wherein said dissolving layer comprises at least one or more ingredients comprising excipients, surfactants, stabilizing agents, emulsifiers, thickeners, preservatives, plasticizers, antimicrobials, water, water soluble polymers, binders, polyethylene oxides, propylene glycols, sweeteners, flavor enhancers, colorants, polyalcohols, and xanthones derived from a mixture of pulp and pericarp of fruit of Garcinia mangostana L. plant.

9. The patch of claim 8, wherein said natural water-soluble polymer is selected from the group consisting of plant-type polymer, microorganism-type polymers and animal-type polymers.

10. The patch of claim 9, wherein said plant-type polymer is selected from the group consisting of gum arabic, gum tragacanth, galactan, guar gum, carob gum, karaya gum, carrageenan, pectin, agar, quince seed or Cydonia oblonga, algae colloids such as brown algae extract, starches such as rice, corn, potato, and wheat, and glycyrrhizic acid.

11. The patch of claim 9, wherein said microorganism-type polymers is selected from the group consisting of xanthan gum, dextran, succinoglucan, and pullulan.

12. The patch of claim 9, wherein said animal-type polymer is collagen, casein, albumin, or gelatin.

13. The patch of claim 8, wherein said water soluble polymer is pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein or mixtures thereof.

14. The patch of claim 8, wherein said water-soluble polymers is a semisynthetic polymer comprising starch-type polymers, cellulosic polymers and alginic acid-type polymers.

15. The patch of claim 8, wherein said water-soluble polymers is a synthetic polymer selected from the group consisting of vinyl polymers, polyoxyethylene-type polymers, acrylic polymers, and cationic polymers, and polyethyleneimine.

16. The patch of claim 8, wherein said thickener is selected from the group consisting of gum arabic, carrageenan, karaya gum, gum tragacanth, carob gum, quince seed or Cydonia oblonga, casein, dextrin, gelatin, sodium pectate, sodium alginate, methyl cellulose, ethyl cellulose, CMC, hydroxy ethyl cellulose, hydroxypropyl cellulose, PVA, PVM, PVP, sodium polyacrylate, carboxy vinyl polymer, locust bean gum, guar gum, tamarind gum, cellulose dialkyl dimethylammonium sulfate, xanthan gum, aluminum magnesium silicate, bentonite, hectorite, AIMg silicate or beagum, laponite, and silicic acid anhydride.

17. The patch of claim 16, wherein said thickener is one or both of methylcellulose or carboxyl methylcellulose in amounts ranging from about 0.01 to about 15 wt %.

18. The patch of claim 8, wherein said surfactant is mono and diglycerides of fatty acids and polyoxyethylene sorbitol esters, Polysorbates, pluronic acid and sodium lauryl sulfate.

19. The patch of claim 18, wherein said surfactant is in an amount of from about 0.5 to about 15 wt %.

20. The patch of claim 8, wherein said stabilizing agent is xanthan gum, locust bean gum, guar gum and carrageenan, sugars, polyols, amino acids or methylamines in amounts ranging from about 0 to about 10 wt %, preferably about 0.1 to about 2 wt % of the film.

21. The patch of claim 8, wherein said emulsifying agent is triethanolamine stearate, quaternary ammonium compounds, acacia, gelatin, lecithin, bentonite, veegum, in amounts ranging from about 0 to about 5 wt %, preferably about 0.01 to about 0.7 wt % of the film.

22. The patch of claim 8, wherein said binding agent is starch, in amounts ranging from about 0 to about 10 wt %, preferably about 0.01 to about 2 wt % of the film.

23. The patch of claim 8, wherein said preservatives or buffers is sodium benzoate.

24. The patch of claim 1, wherein said dissolving layer comprises a hydrocolloid.

25. The patch of claim 24, wherein said hydrocolloid comprises a water soluble natural polysaccharide, pectin, guar gum arabic, tragacanth gum, xanthan gum, gellan sodium salt, propyleneglycol alginate, starches (amylose, amylopectin), modified starches, hydroxyethyl starch, pullulan, carboxymethyl starch, gum ghatti, okra gum, karaya gum, dextrans, dextrins and maltodextrins, konjac, acemannan from aloe, locust bean gum, tara gum, quince seed gum, fenugreek seed gum, scleroglucan, gum arabic, psyllium seed gum, tamarind gum, oat gum, quince seed gum, carrageenans, scleraglucan, succinoglucan, larch arabinogalactan, flaxseed gum, chondroitin sulfates, hyaluronic acid, curdlan, chitosan, deacetylated konjac, or rhizobium gum.

26. The patch of claim 24, wherein said hydrocolloid comprises gelatins, albumins, milk proteins, soy protein, or whey proteins.

27. The patch of claim 24, wherein said hydrocolloid is polyethylene-imine, hydroxyethyl cellulose, sodium carboxymethyl cellulose, carboxymethyl cellulose, hydroxypropyl cellulose, hydroxypropyl methyl cellulose, methyl cellulose, ethyl cellulose, polyacrylic acids, low molecular weight polyacrylamides and their sodium salts (carbomers), polyvinylpyrollidone, polyethylene glycols, polyethylene oxides, polyvinyl alcohols, pluronics, tetronics, or other block co-polymers, carboxyvinyl polymers, or colloidal silicon dioxide.

28. The patch of claim 24, wherein said hydrocolloid is selected from a group consisting of natural seaweeds, natural seed gums, natural plant exudates, natural fruit extracts, biosynthetic gums, gelatines, biosynthetic processed starch or cellulosic materials, alginates, agar gum, guar gum, locust bean gum (carob), carrageenan, tara gum, gum arabic, ghatti gum, Khaya grandifolia gum, tragacanth gum, karaya gum, pectin, arabian (araban), xanthan, gellan, starch, Konjac mannan, galactomannan, funoran, are xanthan, acetan, gellan, welan, rhamsan, furcelleran, succinoglycan, scleroglycan, schizophyllan, tamarind gum, curdlan, pullulan, and dextran.

29. The patch of claim 1, wherein said dissolving layer comprises emulsifying agents, solubilizing agents, wetting agents, plasticizers, active agents, water soluble inert fillers, preservatives, buffering agents, coloring agents, and/or stabilizers.

30. The patch of claim 29, wherein said plasticizer is polyethylene glycol, glycerol, sorbitol, sucrose, corn syrup, fructose, dioctyl-sodium sulfosuccinate, triethyl citrate, trihexyl citrate, tributyl citrate, 1,2-propylenglycol, mono-, di- or triacetates of glycerol, or natural gums.

31. The patch of claim 1, further comprising a film-forming agent comprising polyactide, pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein or mixtures thereof.

32. The patch of claim 31, wherein said film-forming agent is pullulan, in an amount from about 75 wt % to about 99 wt %.

33. The patch of claim 32, wherein said pullulan is in an amount of from about 92 wt % to about 97 wt % of the film.

34. The patch of claim 1, wherein said active material is an antibody, antimicrobial compositions, analgesics, anticoagulants, antiproliferatives, anti-inflammatory compounds, cytokines, cytotoxins, drugs, growth factors, immune response modifiers, interferons, hormones, lipids, demineralized bone or bone morphogenetic proteins, cartilage inducing factors, oligonucleotides polymers, polysaccharides, polypeptides, protease inhibitors, vasoconstrictors or vasodilators, vitamins, minerals, stabilizers, a nucleic acid, a gene construct, an antigen, adjuvants, prodrugs, antibiotics, anti-viral agents, anaesthetics, antineoplastic agents, immunosuppressive agents and immunostimulants; hematopoietic agents and anticoagulants, thrombolytics, anti-histamines, H₂-antagonists, proton pump inhibitors, CNS depressants, CNS stimulants, antiplatelet drugs, chemoattractants, or vaccines.

35. The patch of claim 1, further comprising an adjuvant.

36. The patch of claim 35, wherein said adjuvant is alum, squalene, saponins, allergen, irritant, virosomes, oil-based adjuvants, osteonectin, toll-like-receptor agonists, CpG motifs, all-trans retinoic acid, heat-labile toxin, cholera toxin, a photoimmunomodulator, or an immune response modifier.

37. The patch of claim 1, wherein said dissolving layer further comprises material that alters the barrier function of the skin.

38. The patch of claim 37, wherein said material is an antimetabolite, capsaicin, hydrocortisone, or comprises a permeation enhancer.

39. A vaccine delivery patch, comprising:
a dissolvable layer containing an antigen, wherein said layer was produced by drying a formulation comprised of 2.05% pullulan, 0.086% β-carrageenan, 0.014% polysorbate A and deionized water; and
an adhesive backing or cover.

40. A method of transdermally vaccinating an animal, comprising the steps of:
ablating partially or completely an area of the stratum corneum of the animal; and
applying the patch of claim 1 to said area.

41. A method of delivering a pharmaceutical substance to the skin of an animal, comprising the steps of:
ablating partially or completely an area of the stratum corneum of the animal; and
applying the patch of claim 1 to said area.
